(19) United States
(12) Patent Application Publication
(10) Pub. No.: US 2026/0001827 A1
(43) Pub. Date: Jan. 1, 2026

(54) SYSTEMS AND PROCESSES FOR THE PRODUCTION OF MTBE AND MALEIC ANHYDRIDE FROM C4 HYDROCARBONS THEREFROM

(71) Applicant: SABIC GLOBAL TECHNOLOGIES B.V., BERGEN OP ZOOM (NL)

(72) Inventors: Mohammad Basheer AHMED, Bangalore (IN); Abdulrahman Shahid SARANG, Bangalore (IN); Hatem Abdallah BELFADHEL, Bangalore (IN)

(73) Assignee: SABIC GLOBAL TECHNOLOGIES B.V., BERGEN OP ZOOM (NL)

(21) Appl. No.: 18/992,831

(22) PCT Filed: Jul. 6, 2023
(86) PCT No.: PCT/EP2023/068770

§ 371 Date: Jan. 9, 2025

(30) Foreign Application Priority Data
Jul. 9, 2022 (IN) 202241039479

(51) Int. Cl. C07C 41/06; C07C 43/04; C07D 307/46

(52) U.S. Cl. CPC C07C 41/06; C07C 43/046; C07D 307/46

(57) ABSTRACT

A system and method for production of MTBE, maleic anhydride, n-butane and/or olefins, is disclosed. The system includes a feed preparation unit, a deisobutanizer, an isobutane dehydrogenation unit, a steam cracker and a hydrogenation unit, as well as either or both of an MTBE synthesis unit or a maleic anhydride unit.

### CROSS REFERENCE TO RELATED APPLICATIONS

None.

### BACKGROUND OF THE INVENTION

### A. Field of the Invention

The invention generally concerns systems and processes for the production of C4 hydrocarbon products, and Methyl-Tertiary-Butyl-Ether (MTBE) and maleic anhydride (MA) produced therefrom. The present invention describes the development of a process scheme to produce MTBE and MA from a C4 hydrocarbon stream containing C3 hydrocarbons, isobutane (iC4), normal butane (n-butane or nC4 herein) and C5 or higher hydrocarbons (C5+) typically from a stream containing 2 to 5 wt. % C3 hydrocarbons, 25 to 35 wt. % isobutane, 65 to 75 wt. % n-butane and 1% to 2 wt. % C5+ hydrocarbons. This is accomplished by separating the C4 hydrocarbon stream in a deisobutanizing unit to produce an isobutane rich stream containing from >90 wt. % to 100 wt. % iso-butane and an n-butane rich stream containing >90 wt. % to 100 wt. % n-butane. These isobutane and n-butane rich streams are prepared by distilling in a distillation unit, preferably a deisobutanizing unit, a fresh C4 feed, a recycle stream after a hydrogenation process, or any other C4-containing stream that contains n-butane and isobutane as components. The overhead stream of the distillation unit forms a rich isobutane stream used to produce MTBE via dehydrogenation or etherification, whereas the rich nC4 stream is used to produce MA, fed to a steam cracker as a steam cracker feed, or both. A bottom stream from the deisobutanizer unit contains C5+ hydrocarbons which may be further processed, e.g., by recycling to a steam cracker.

### B. Description of Related Art

Methyl tert-butyl ether (MTBE) is a flammable liquid used as an additive for unleaded gasoline to increase octane and oxygen levels and to reduce pollution emissions. MTBE is prepared from isobutane or isobutylene which is preferably from crude steam cracker or fluidized catalytic cracker (FCC) C4 hydrocarbon stream. The C4 hydrocarbon stream contains a mixture of C4 hydrocarbons including butane and isobutane from a fresh C4 stream, a hydrogenated C4 recycle stream from a steam cracker after hydrogenation, or from a fluid catalytic cracking process, and the isobutane must be isolated from the C4 hydrocarbon stream for MTBE synthesis, or it must be prepared from n-butane via isomerization in a butane isomerization unit, also known as a butamer, to yield isobutane.

Maleic anhydride (MA), on the other hand, is prepared from n-butane, so n-butane must be isolated for production of this product.

Overall, while the technologies of producing MTBE and/or maleic anhydride (MA) exist, they can be energy inefficient and expensive.

Thus, while technologies for producing MTBE and maleic anhydride (MA) exist, new and more efficient systems and processes for preparing these products are desired. It would be especially beneficial to produce these products from C4 streams without any need to isomerize n-butane to prepare MTBE.

### SUMMARY OF THE INVENTION

A discovery has been made that provides a solution to at least one of the problems associated with production of MTBE and/or maleic anhydride. An aspect relates to a method for producing MTBE and/or maleic anhydride from C4 streams containing mixtures of C4 hydrocarbons that contain n-butane and isobutane.

Aspects of the invention relate to a method for preparing methyl tert-butyl ether (MTBE). The method includes the steps of separating from a hydrocarbon feed a stream having a boiling point of less than 200° C., a bottom stream comprising fuel and pitch, and a steam cracker feed; feeding the steam cracker stream to a steam cracker and reacting under conditions to form a reaction product comprising hydrogen, methane, ethylene, propylene, 1,3-butadiene, 1-butene, 2-butene, isobutylene, isobutane, n-butane, pygas, fuel oil and unreacted ethane, propane and C4 paraffins; separating lights (gases), olefins, pygas, fuel oil from the reaction product to form C4 olefins (1,3 butadiene, 1-butene, 2-butene, isobutylene) and C4 paraffins (isobutane and n-butane), designated crude steam-cracked C4. The 1,3 butadiene, isobutylene, which is used in MTBE production, and 1-butene are separated from the C4 olefin stream and may be recovered as product, while the remainder of the stream, designated as Raff-3, contains 2-butene, isopropane and n-butane. The Raff-3 stream is fed to a hydrogenation unit to form C4 paraffins and produce a hydrogenated C4 stream comprising primarily a combination of isobutane and n-butane. The hydrogenated C4 stream is combined with a fresh C4 hydrocarbon stream to form a combined C4 paraffin stream, and the combined C4 paraffin stream is fed to a deisobutanizer unit to form an isobutane stream, a normal butane stream containing the n-butane, and optionally a bottom stream comprising C5+ hydrocarbons. The normal butane stream is a side draw stream from the deisobutanizer unit and is rich in n-butane, and is fed to an isomerization unit to convert n-butane to isobutane under conditions sufficient to effect the isomerization of n-butane to isobutane. Effluent from the isomerization reactor is recycled back to the deisobutanizer (DIB) unit for separation. The deisobutanizer top stream is rich in isobutane and is fed to an isobutane dehydrogenation (IBDH) unit, where isobutylene is produced under conditions sufficient to convert isobutane to isobutylene. A crude C4 stream is separated from a steam cracker and contains 1,3 butadiene, isobutylene, 1-butene, 2-butene and saturated C4 hydrocarbons (i.e., isobutane and n-butane). The 1,3-butadiene is removed from the crude C4 stream to produce a stream designated as Raff-1, and the Raff-1 stream is fed to an MTBE unit. Raff-1 is combined with IBDH effluent and methanol and produce MTBE. The remaining stream after isobutylene conversion may be fed to an 1-butene recovery unit and the remainder of the stream may be hydrogenated. The hydrogenated stream contains a mixture of isobutane and n-butane, and may be sent to the steam cracker to produce olefins, or, optionally, fed to the DIB unit to produce isobutene for MTBE production. An overhead vent stream produced from DIB unit contains C3 hydrocarbons and a bottom stream produced from the DIB unit contains primarily C5+ hydrocarbons. The C3 hydrocarbon and C5+ hydrocarbon streams may be processed in a stream cracker to produce various products including C2-C3 olefins, methane, hydrogen, and heavier products such as pygas and fuel oil.

Aspects also relate to a method for preparing methyl tert-butyl ether (MTBE). The method includes the steps of separating from a hydrocarbon feed stream having a boiling point of less than 200° C., a bottom stream comprising fuel and pitch, and a crude C4 hydrocarbon stream comprising 1,3 butadiene, isobutylene, 1-butene, 2-butene and C4 paraffins (isobutane and n-butane). The complete crude C4 hydrocarbon stream from the steam cracker is hydrogenated to form C4 paraffins and yield a hydrogenated C4 stream primarily comprising isobutane and n-butane; combining the hydrogenated C4 stream with the fresh C4 hydrocarbon stream (which may, for example, contain 2 to 5 wt. % C3 hydrocarbons, 25 to 35 wt. % isobutane, 65 to 75 wt. % n-butane and 1% to 2 wt. % C5+) to form a combined C4 paraffin stream; feeding the combined C4 paraffin stream into a deisobutanizer unit to form an isobutane stream, a normal butane stream containing the normal C4 butane, and optionally a bottom stream comprising C5+ hydrocarbons. The remainder of the process is as described above.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment or aspect discussed herein can be combined with other embodiments or aspects discussed herein and/or implemented with respect to any method or composition of the invention, and vice versa. Furthermore, compositions of the invention can be used to achieve methods of the invention.

The following includes definitions of various terms and phrases used throughout this specification.

The term “C# hydrocarbons”, wherein “#” is a positive integer, is meant to describe all hydrocarbons having # carbon atoms. Moreover, the term “C#+ hydrocarbons” is meant to describe all hydrocarbon molecules having # or more carbon atoms. Accordingly, the term “C2+ hydrocarbons” is meant to describe a mixture of hydrocarbons having 2 or more carbon atoms. The term “C2+ alkanes” accordingly relates to alkanes having 2 or more carbon atoms.

“Cracking” refers to a process involving decomposition and molecular recombination of organic compounds to produce a greater number of molecules than were initially present. In cracking, a series of reactions take place accompanied by a transfer of hydrogen atoms between molecules. For example, naphtha may undergo a thermal cracking reaction to form ethene and hydrogen, methane, ethylene, propylene, 1,3 butadiene, C4 olefins, C4 paraffins, BTX and PFO, as well as other products.

“Hydrocarbons” are generally defined as molecules formed primarily by carbon and hydrogen atoms. Hydrocarbons may also include other elements such as, but not limited to, halogens, metallic elements, nitrogen, oxygen, and/or sulfur. Hydrocarbon fluids may include, entrain, or be entrained in non-hydrocarbon fluids such as hydrogen, nitrogen, carbon monoxide, carbon dioxide, hydrogen sulfide, water, and/or ammonia.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The systems and processes of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the systems and methods of the present invention are their abilities to produce olefin products (e.g., ethylene) in a cost and energy efficient manner by having an ethane steam cracker unit capable of receiving ethane from a mixed feed steam cracker unit and feeding the C2+ products produced by the ethane steam cracker unit to the mixed feed steam cracker unit.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 illustrates an embodiment of a system to produce MTBE, 1-butene and n-butane without hydrogenation of the crude C4 stream.

FIG. 2 illustrates an embodiment of a system to produce MTBE, 1-butene and n-butane with complete hydrogenation of the crude C4 stream.

FIG. 3 illustrates an embodiment of a system to produce maleic anhydride from n-butane without hydrogenation of the crude C4 stream.

FIG. 4 illustrates an embodiment of a system to produce maleic anhydride from n-butane with complete hydrogenation of the crude C4 stream.

FIG. 5 illustrates an embodiment of a system to produce MTBE, 1-butene and n-butane utilizing a steam cracker without hydrogenation of the crude C4 stream.

FIG. 6 illustrates an embodiment of a system to produce MTBE, 1-butene and n-butane utilizing a steam cracker with complete hydrogenation of the crude C4 stream.

While the invention is susceptible to various modifications and alternative forms, specific embodiments thereof are shown by way of example in the drawings. The drawings may not be to scale.

### DETAILED DESCRIPTION OF THE INVENTION

A discovery has been made that provides a solution to at least one of the problems associated with preparing MTBE and maleic anhydride. In one aspect, a crude mixed C4 hydrocarbon feed can be fed to a deisobutanizer unit to produce an isobutane stream, an n-butane stream, and a C5+ stream. The crude C4 stream is produced from a steam cracker and is preferably provided by feeding crude oil to a feed preparation unit, wherein it is separated to produce a gas and liquid stream, and a cracker bottom stream containing fuels and pitch.

The isobutane stream is fed to an isobutane dehydrogenation unit to produce isobutene, which is then fed to an MTBE synthesis unit where it is reacted with methanol to produce MTBE. A portion of the rich isobutane stream may be fed to a polyethylene plant.

The n-butane stream can be recovered as product, or it may be fed to a maleic anhydride synthesis unit to be reacted under conditions to produce maleic anhydride, or, alternatively, or in combination, to a steam cracker to enhance production of ethylene and other chemical products.

Conditions in the maleic anhydride unit include temperatures of 375° C. to 425° C. Typically conversions rates are 80-86 wt. % n-butane conversion, which yields 50-60 wt. % of maleic anhydride. Maleic anhydride produced from n-butane purity >95 wt. %) by various commercially technologies using fixed and fluidized bed reactors. As an example, using a catalyst like vanadyl pyrophosphate oxide (VO)₂P₂O₇ with trimethyl phosphate as a promotor can yield 90 wt. % of MA, and will consume 1.1 to 1.2 tons of n-butane per ton of MA. Reaction is oxidative and exothermic, need to remove heat from reactor.

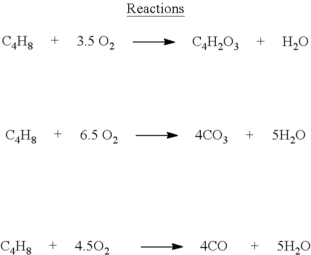

The effluent from reactor comprise gaseous maleic anhydride, water, acrylic acid, acetic acid, CO, CO₂, O₂, N₂ and unreacted n-butane, cooled to 120° C. in series of exchangers. Then the cooled mixture is sent to a solvent absorption system to absorb MA. Exemplary suitable solvent include, dialkyl phthalates, dimethylbenzophenone and dichlorodiphenyloxide. The MA subsequently stripped from absorption liquid to produce crude MA. The solvent extraction system recover 90-96 wt. % of maleic anhydride with >99 wt. % purity from off gas effluent.

The gas and liquid stream from the feed preparation unit is sent to a steam cracker where it is reacted under thermal conditions to yield olefins and other products.

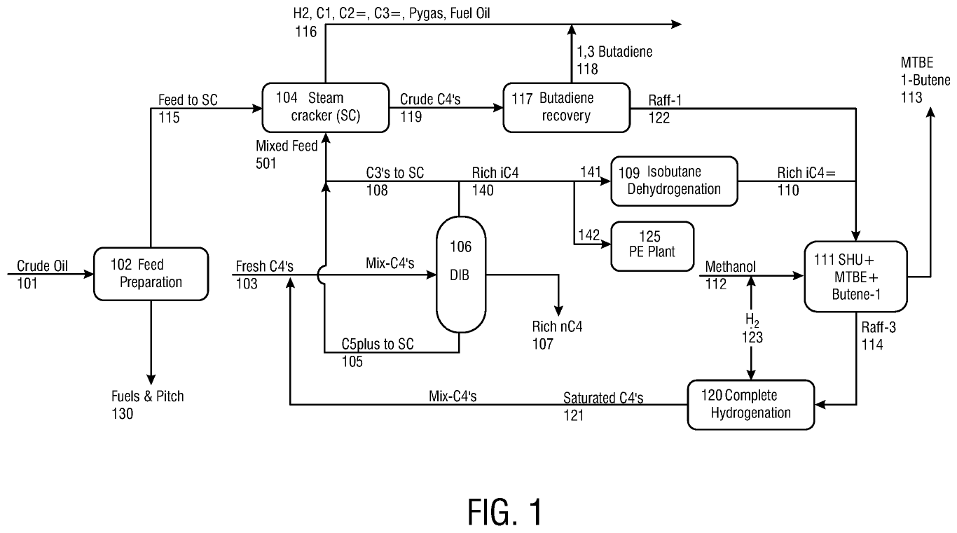

Referring to FIG. 1, system 100 for producing MTBE is described. System 100 can include a feed separation unit 102, a steam cracking unit 104, a butadiene recovery unit 117, selective hydrogenation, MTBE, butene-1 unit 111, a deisobutanizer unit 106, an isobutane dehydrogenation unit 109, polyethylene plant 125 and complete hydrogenation unit 120. Crude oil 101 enters feed separation unit 102, produce steam cracker feed 115 and fuel and pitch 130. First fresh C4 stream 103 combined with hydrogenated C4's from full hydrogenation unit 120 is fed to deisobutanizer 106, where vent stream 108 is rich in C3's and to a steam cracker. The overhead liquid stream containing rich isobutane (iC4) 141 & 142, a second stream (side draw) containing rich n-butane 107, and a third stream containing C5+ hydrocarbons 105 is sent to steam cracker 104. The first stream containing isobutanes is fed to isobutane dehydrogenation unit 109 where the isobutane reacts with a catalyst to form isobutene (iC4=) product 110. The isobutene product from butadiene recovery unit 109 combined with Raff-1 from butadiene recovery unit 117 unit is fed to MTBE synthesis unit 111 and mixed with methanol from methanol stream 112 which is fed into MTBE synthesis unit under conditions sufficient to produce MTBE. 1-butene product produced from raffinate stream of MTBE unit. Raff-3 stream 114 from MTBE synthesis unit 111 is hydrogenated completely in complete hydrogenation unit 120 to produce saturated C4's stream 121, which is recycled to deisobutanizer 106.

The MTBE and 1-butene is separated from unreacted isobutene, 1-butene, 2-butene and other paraffin C4 hydrocarbons to form MTBE and 1-butene product stream 113, with the remainder exiting the MTBE unit as an MTBE effluent stream 114 (Raff-3).

Gas and liquids stream 115 contains hydrocarbons other than C4 hydrocarbons and is fed to steam cracker 104 wherein the feed is thermally cracked in steam cracker 104 to produce olefins and other products. Olefins and other steam cracker products recovered in stream 116. The crude C4 hydrocarbon stream 119 from steam cracker 104 may be fed to Butadiene recovery unit 117 wherein the 1,3 butadiene product is separated as a product stream 118 and combined with olefin product stream 116, and preferably is collected for use. The Raff-1 stream 122, after butadiene recovery is combined with rich isobutylene stream 110 from unit 109 send to unit 111, where MTBE and 1-butene product produced 113.

The first stream 140 from deisobutanizer can be split in to stream 141 and stream 142, whereas stream 141 send to isobutane dehydrogenation unit 109 and a small portion of 140 as a product stream 142 to polymer plant (PE).

The second stream 107 from deisobutanizer is rich in nC4 as a product

Crude oil 101 can be the petroleum extracted from geologic formations in its unrefined form. The term crude oil can also include petroleum that has been subjected to water-oil separations and/or gas-oil separation and/or desalting and/or stabilization. Non-limiting examples of crude oil include Arabian Heavy, Arabian Light, other Gulf crudes, Brent, North Sea crudes, North and West African crudes, Indonesian, Chinese crudes, West Texas crude, and mixtures thereof, but also shale oil, tar sands, gas condensates and bio-based oils. The crude oil used as feed to the process of the present invention preferably is conventional petroleum having an API gravity of more than 20° API as measured by the ASTM D287 standard. In one aspect, the crude oil used in the process of the present invention is a light crude oil having an API gravity of more than 30° API. In another aspect, the crude oil used in the process of the present invention can include Arabian Light Crude Oil. Arabian Light Crude Oil typically has an API gravity of between 32-36° API and a sulfur content of between 1.5-4.5 wt. %.

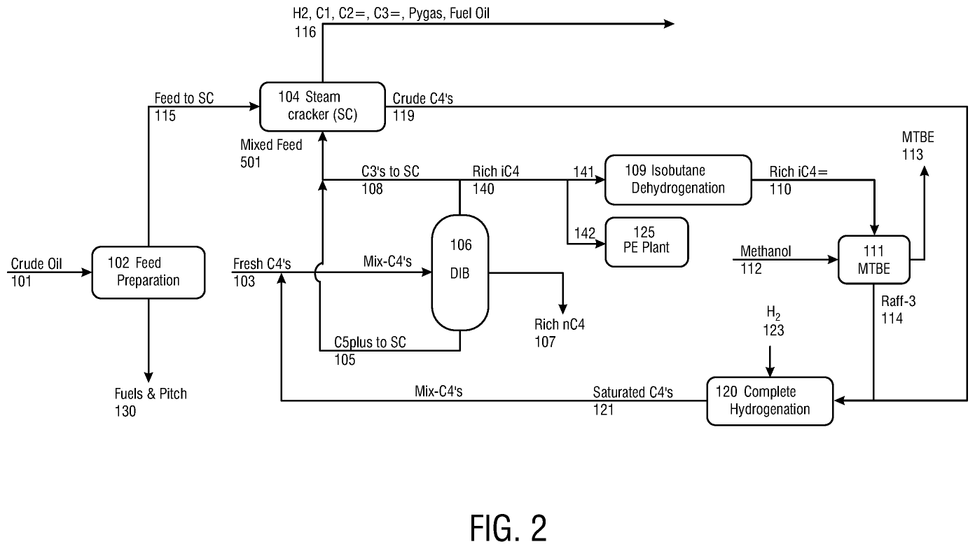

Referring to FIG. 2, an alternative process is shown wherein crude C4 stream 119 is fed directly to hydrogenation unit 120, and saturated C4 stream 121 exits hydrogenation unit 120 by hydrogen stream 123 and is combined with fresh C4 stream 103. In this embodiment, methanol is combined with isobutene stream 110 and the combined isobutene/methanol stream 201 is fed to MTBE synthesis unit 111 where it is reacted to produce MTBE.

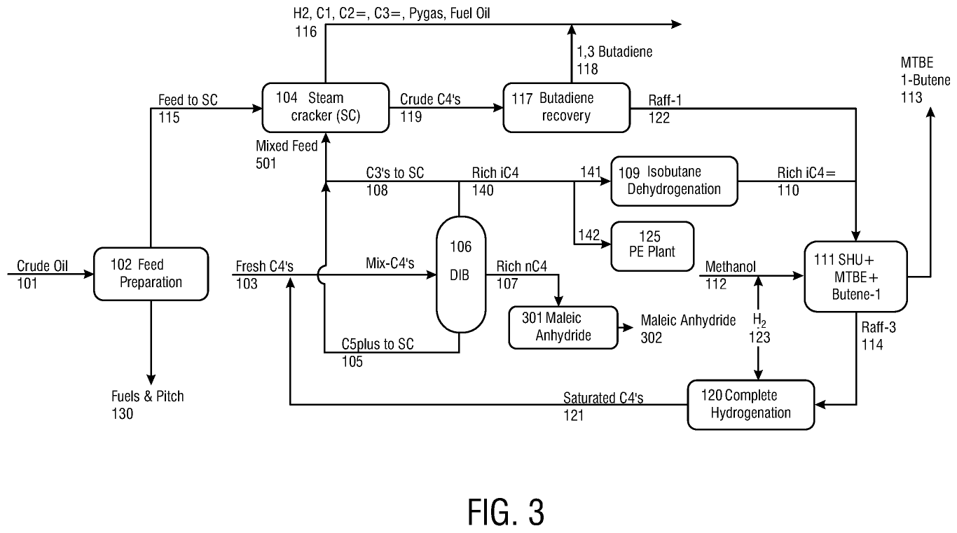

FIG. 3 is similar to FIG. 1, except that the second stream containing n-butane 107 is fed to maleic anhydride synthesis unit 301 to produce maleic anhydride product 302.

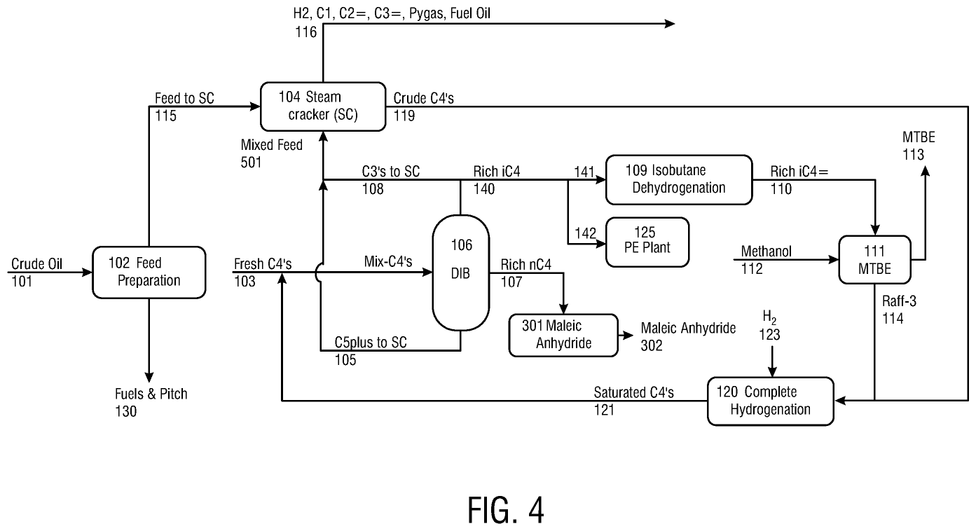

FIG. 4 is similar to FIG. 2, except that second stream containing n-butane 107 is fed to maleic anhydride synthesis unit 301 to produce maleic anhydride product 302.

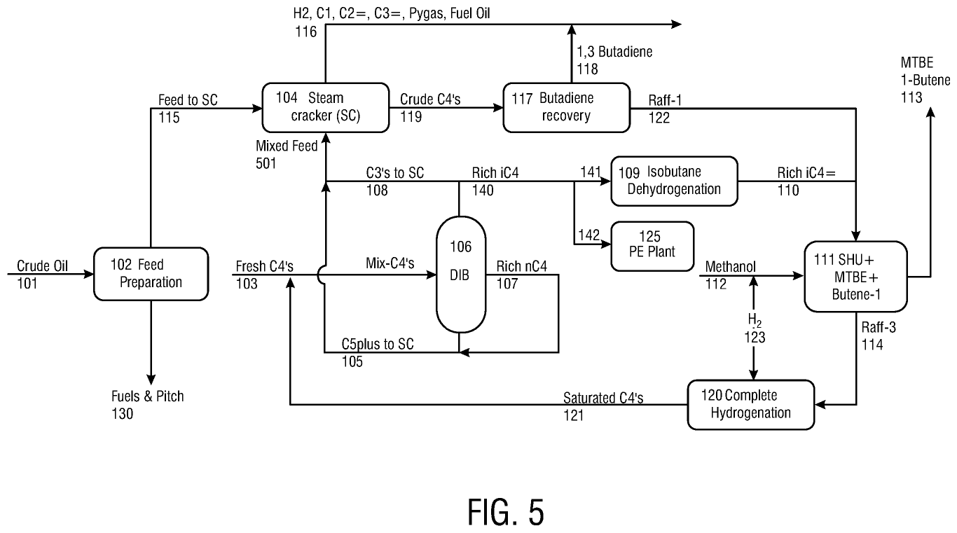

FIG. 5 is similar to FIG. 1, except that the second stream containing n-butane 107 is combined with C5+ stream 105 and C3's stream 108 to form mixed steam cracker feed stream 501 which is fed to steam cracker 104. Gas and liquid stream 115 may be combined with mixed steam cracker feed stream 501 prior to entering steam cracker 104.

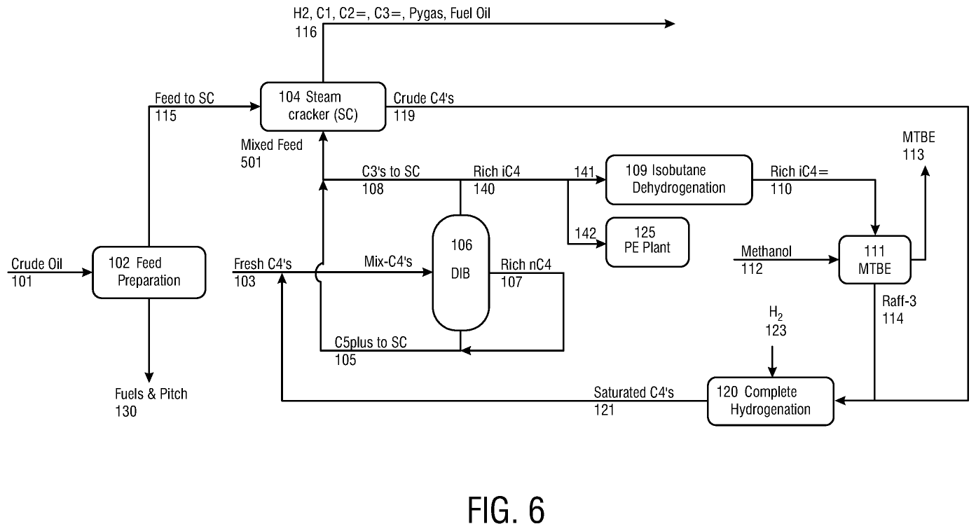

FIG. 6 is similar to FIG. 2, except that the second stream containing n-butane 107 is combined with C5+ stream 105 and C3's stream 108 to form mixed steam cracker feed stream 501 which is fed to steam cracker 104. Gas and liquid stream 115 may be combined with mixed steam cracker feed stream 501 prior to entering steam cracker 104.

The hydrogenation reaction carried out at pressure from 22 to 32 bara and temperature from 58 to 125 is ° C. . . . The hydrogen to feed ratio of 0.01 to 0.03 wt./wt. A portion of the H₂ is separated from cold box separator flash drum. Another portion of H₂ and methane may be separated from a demethanizer unit.

The first stream containing isobutanes is fed to isobutane dehydrogenation unit 109 where the isobutane reacts with a catalyst to form isobutene (iC4=) product 110. The isobutene product from 109 combined with Raff-1 from 117 unit is fed to MTBE synthesis unit 111 and mixed with methanol from methanol stream 112 which is fed into MTBE synthesis unit under conditions sufficient to produce MTBE. 1-butene product produced from raffinate stream of MTBE unit. The Raff-3 stream 114 from unit 111 is hydrogenated completely in 120 to produce saturated C4's stream 121, which is recycled to deisobutanizer.

The dehydrogenation reaction is an equilibrium reaction favoring low pressures. The reaction is highly endothermic require large amount of heat to be supplied to radial flow moving bed reactor in series or fixed bed with stable heterogeneous catalyst is required. The isobutane dehydrogenation reaction carried out at pressure from 0.5 to 15 psia and temperature from 550 to 700° C. The conversion of isobutane range from 45 to 65 wt. % with a selectivity of 87 to 95 wt. %. The following reaction occur in isobutane dehydrogenation:

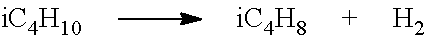

The MTBE and 1-butene is separated from unreacted isobutene, 1-butene, 2-butene and other paraffin C4's to form MTBE and 1-butene product stream 113, with the remainder exiting the MTBE unit as an MTBE effluent stream 114 (Raff-3). A typical content of a Raff-3 stream is provided in the table below:

| Raff -3 composition | wt. % |
|---|---|
| C3's | to 1% |
| Isobutene | 0.2 to 0.8% |
| 1-butene | 1 to 3% |
| Trans-2butene | 48 to 55% |
| Cis-2butene | 15 to 25% |
| iC4 | 1 to 3.5% |
| nC4 | 17 to 28% |

Gas and liquids stream 115 other than C4's is fed to steam cracker 104 wherein the feed cracked thermally in steam cracker 104 to produce olefins and other products. Olefins and other steam cracker products recovered in stream 116. The crude C4 hydrocarbon stream 119 from steam cracker 104 may be fed to Butadiene recovery unit 117 wherein 1,3 butadiene product is separated as a product stream 118 and combined with olefin product stream 116 and collected for use. The Raff-1 stream 122, after butadiene recovery is combined with rich isobutylene stream 110 from unit 109 send to unit 111, where MTBE and 1-butene product produced 113. The Raff-1 stream may comprise, for example, may preferably contain 45 wt. % to 58 wt. % of isobutylene, 12 wt. % to 27 wt. % of 1-butene, 5 wt. % to 16 wt. % of 2-butenes (both cis & trans 2-butenes), 5 wt. % to 12 wt. % of isobutane, 2 wt. % to 8 wt. % of n-butane and <1% 1,3 butadiene.

In the steam cracking process, the saturated hydrocarbons are broken down into smaller, often unsaturated, hydrocarbons such as ethylene, propylene, 1,3 butadiene, butenes, pygas etc., by diluting the mixed hydrocarbon feed with steam and heating the mixture in a furnace in the absence presence of oxygen. The steam cracking reaction can have a residence times of 50-1000 milliseconds. Steam cracker effluent is separated via downstream separation section (not shown), where steam cracked products separated from unreacted components. Such fractionation/separation units are well known in the art.

In a separation unit downstream from the steam cracker, the steam cracker effluent is cooled from 850° C. to 180° C. via series of transfer line exchanges, where high pressure steam is produced. Cooled effluent is send to quench oil or primary fractionation system to recover fuel oil. The overhead stream of the primary fractionator is sent to a quench water tower, where the effluent further cools via direct contact with water. The overhead stream of the quench tower contains hydrocarbons boiling at about 30° C. to 45° C., whereas a bottom stream contains pygas related components boils at about 70° C. to 85° C. The bottom of the quench section contains settler to separate pygas from water. Water is used as heating media and recycled back to quench tower.

The overhead effluent send to cracked gas compressor, where it is compressed to 30 bar to 45 bar. After a 3^{rd} stage compressor CO₂ and H₂S may be separated in caustic tower, CO other are impurity level components. The remaining stream may be sent to 4th and 5th stage of compressor to achieve the pressure.

After compression, effluent is dried in dryer, and cooled by exchange heat from various streams. A portion of H₂ may be separated from cold box separator flash drum. The remainder of the H₂ and methane separated from demethanizer overhead.

The demethanizer bottom stream is sent to a deethanizer unit, where C2 hydrocarbons are separated into acetylene, ethylene and ethane. A bottom stream of the deethanizer unit contains C3+ hydrocarbons and may sent to a depropanizer unit. The C2 stream from deethanizer may be sent to to C2 Acetylene converted in acetylene reactor to ethylene and ethane, where ethylene is recovered in C2 splitter column, bottom of the C2 splitter is ethane, where recycle back to steam cracking furnace.

The depropanizer overhead sent to MAPD may converted in a C3 acetylene reactor to propane and propylene. Propylene may be recovered via C3 splitter column as product, and the bottom stream of the C3 splitter contains propane and may be recycled back to the steam cracking furnace.

The depropanizer bottoms may be fed to a debutanizer unit to separate C4 stream from C5+. Where debutanizer overhead contains crude C4 stream comprise, limited C3, C5's and C4 acetylene and majority of 1,3 butadiene, isobutylene, 1-butene, 2-butene (cis and trans), n-C4 and iC4. This stream called crude C4 stream.

The C5 plus stream called pygas is hydrogenated in gasoline hydrogenation unit, send to a depentanizer unit to separate C5 hydrocarbons from a C6 stream. Overhead stream of the depentanizer unit is mostly C5 paraffins (nC5 & isoC5) recycled back to steam cracking furnace.

The C6+ stream is further processed in hydro dealkylation unit to convert other C6+ component in to benzene in presence of hydrogen. A final benzene product may recovered from hydrodealkylation unit.

The fuel oil or gas oil product may be recovered from quench oil and quench water tower bottoms.

C9+ hydrocarbons may be recovered from benzene plant.

The first stream 140 from deisobutanizer can be split in to stream 141 & stream 142, whereas stream 141 send to isobutane dehydrogenation unit 109 and a small portion of 140 as a product stream 142 to polymer plant (PE).

Aspects also relate to a method for preparing methyl tert-butyl ether (MTBE), the method including the steps of separating from a hydrocarbon feed a gaseous stream having a boiling point of less than 200° C., a bottom stream comprising fuel and pitch, and a crude C4 hydrocarbon stream comprising C4 hydrocarbons from, wherein the crude C4 hydrocarbon stream comprises normal butanes and isobutane; feeding the gaseous stream to a steam cracker and reacting the gaseous stream in the steam cracker with a steam cracking catalyst to form a reaction product comprising C4 olefins and unreacted C4 paraffins; separating from the reaction product the C4 olefins and the unreacted C4 paraffins to form a crude steam-cracked C4 stream containing the C4 olefins and the unreacted C4 paraffins, and feeding the crude steam-cracked C4 stream to a hydrogenation unit; hydrogenating the crude steam-cracked C4 stream in the hydrogenation unit to hydrogenate the C4 olefins to form C4 paraffins and produce a hydrogenated C4 stream comprising isobutane and normal butane; combining the hydrogenated C4 stream with the crude C4 hydrocarbon stream to form a combined C4 paraffin stream; feeding the combined C4 paraffin stream into a deisobutanizer unit to form an isobutane stream, a normal butane stream containing the normal C4 butane, and a bottom stream comprising C5+ hydrocarbons; feeding the isobutane stream to an isobutane dehydrogenation unit under process conditions sufficient to dehydrogenate the isobutane to form a C4 hydrocarbon mixture comprising isobutene and unreacted C4 hydrocarbons; feeding the C4 hydrocarbon mixture to an MTBE synthesis unit; and reacting the isobutene with methanol in the presence of a catalyst in the MTBE synthesis unit to form MTBE; separating the methyl tertiary butyl ether from the unreacted portion of the distillate stream, the unreacted portion comprising isobutane and 1-butene; combining the normal butane stream and the bottom stream comprising C5+ hydrocarbons to form a combined C4/C5+ stream and feeding the combined C4/C5+ stream to the steam cracker. Preferably the hydrocarbon feed is crude oil. Reaction conditions in the isobutane dehydrogenation unit include a pressure from 0.5 to 15 psia and temperature from 550° C. to 700° C. The conversion of isobutane range from 45 to 65 wt. % with a selectivity of 87 to 95 wt. %.

Aspects provide a method for preparing methyl tert-butyl ether (MTBE), the method including the steps of separating from a hydrocarbon feed a gaseous stream having a boiling point of less than 200° C., a bottom stream comprising fuel and pitch, and a crude C4 hydrocarbon stream comprising C4 hydrocarbons from, wherein the crude C4 hydrocarbon stream comprises normal butanes and isobutane; feeding the gaseous stream to a steam cracker and reacting the gaseous stream in the steam cracker with a steam cracking catalyst to form a reaction product comprising olefins, wherein the olefins comprise C4 olefins; separating the C4 olefins to form a C4 olefin stream and feeding the C4 olefin stream into a hydrogenation unit; hydrogenating the C4 olefin stream with hydrogen under conditions such that the C4 olefins are hydrogenated to form C4 paraffins comprising isobutane and normal butane; combining the C4 paraffins with the fresh C4 hydrocarbon stream to form a combined C4 stream; feeding the combined C4 hydrocarbon stream to a deisobutanizer unit to form an isobutane stream, a normal butane stream containing the normal C4 butanes, and a bottom stream comprising C5+ hydrocarbons; feeding the isobutane stream to an isobutane dehydrogenation unit under process conditions sufficient to dehydrogenate the isobutane to form a C4 hydrocarbon mixture comprising isobutene and unreacted C4 hydrocarbons; feeding the C4 hydrocarbon mixture to an MTBE synthesis unit; and reacting the isobutene with methanol in the presence of a catalyst in the MTBE synthesis unit to form MTBE; separating the methyl tertiary butyl ether from the unreacted portion of the distillate stream, the unreacted portion comprising isobutane and 1-butene; and combining the normal butane stream and the bottom stream comprising C5+ hydrocarbons to form a combined C4/C5+ stream and feeding the combined C4/C5+ stream to the steam cracker. Preferably the hydrocarbon feed is crude oil. Reaction conditions in the isobutane dehydrogenation unit include a pressure from 0.5 to 15 psia and temperature from 550° C. to 700° C. Reaction conditions during hydrogenation include a pressure in the range of from 22 to 32 bara and temperature from 50 to 125° C. The hydrogen to feed ratio of 0.01 to 0.03 wt./wt.

Non-limiting embodiments of the invention will now be described. Embodiment 1 is a method for preparing methyl tert-butyl ether (MTBE). The method includes the steps of separating from a hydrocarbon feed a gaseous stream having a boiling point of less than 200° C., a bottom stream containing fuel and pitch, and a crude C4 hydrocarbon stream containing C4 hydrocarbons from, wherein the crude C4 hydrocarbon stream contains normal butanes and isobutane; feeding the gaseous stream to a steam cracker and reacting the gaseous stream in the steam cracker with a steam cracking catalyst to form a reaction product containing C4 olefins and unreacted C4 paraffins; separating from the reaction product the C4 olefins and the unreacted C4 paraffins to form a crude steam-cracked C4 stream containing the C4 olefins and the unreacted C4 paraffins, and feeding the crude steam-cracked C4 stream to a hydrogenation unit; hydrogenating the crude steam-cracked C4 stream in the hydrogenation unit to hydrogenate the C4 olefins to form C4 paraffins and produce a hydrogenated C4 stream containing isobutane and normal butane; combining the hydrogenated C4 stream with the crude C4 hydrocarbon stream to form a combined C4 paraffin stream; feeding the combined C4 paraffin stream into a deisobutanizer unit to form an isobutane stream, a normal butane stream containing the normal C4 butane, and optionally a bottom stream containing C5+ hydrocarbons; feeding the isobutane stream to an isobutane dehydrogenation unit under process conditions sufficient to dehydrogenate the isobutane to form a C4 hydrocarbon mixture containing isobutene and unreacted C4 hydrocarbons; feeding the C4 hydrocarbon mixture to an MTBE synthesis unit; and reacting the isobutene with methanol in the presence of a catalyst in the MTBE synthesis unit to form MTBE; and separating the methyl tertiary butyl ether from the unreacted portion of the distillate stream, the unreacted portion containing isobutane and 1-butene. Embodiment 2 is the method of embodiment 1, wherein the normal butane stream is fed into a maleic anhydride unit. Embodiment 3 is the method of embodiment 1, wherein the hydrocarbon feed is crude oil. Embodiment 4 is the method of embodiment 1, wherein the reaction conditions in the isobutane dehydrogenation unit include a pressure from 0.5 to 15 psia and temperature from 550° C. to 700° C. Embodiment 5 is the method of embodiment 1, wherein the reaction conditions during hydrogenation include a pressure in the range of from 22 to 32 bara and temperature from 50 to 125° C. The hydrogen to feed ratio of 0.01 to 0.03 wt./wt. Embodiment 6 is a method for preparing methyl tert-butyl ether (MTBE), the method including the steps of separating from a hydrocarbon feed a gaseous stream having a boiling point of less than 200° C., a bottom stream containing fuel and pitch, and a crude C4 hydrocarbon stream containing C4 hydrocarbons from, wherein the crude C4 hydrocarbon stream contains normal butanes and isobutane; feeding the gaseous stream to a steam cracker and reacting the gaseous stream in the steam cracker with a steam cracking catalyst to form a reaction product containing olefins, wherein the olefins contains C4 olefins; separating the C4 olefins to form a C4 olefin stream and feeding the C4 olefin stream into a hydrogenation unit; hydrogenating the C4 olefin stream with hydrogen under conditions such that the C4 olefins are hydrogenated to form C4 paraffins containing isobutane and normal butane; combining the C4 paraffins with the fresh C4 hydrocarbon stream to form a combined C4 stream; feeding the combined C4 hydrocarbon stream to a deisobutanizer unit to form an isobutane stream, a normal butane stream containing the normal C4 butanes, and a bottom stream containing C5+ hydrocarbons; feeding the isobutane stream to an isobutane dehydrogenation unit under process conditions sufficient to dehydrogenate the isobutane to form a C4 hydrocarbon mixture containing isobutene and unreacted C4 hydrocarbons; feeding the C4 hydrocarbon mixture to an MTBE synthesis unit; reacting the isobutene with methanol in the presence of a catalyst in the MTBE synthesis unit to form MTBE; and separating the methyl tertiary butyl ether from the unreacted portion of the distillate stream, the unreacted portion containing isobutane and 1-butene. Embodiment 7 is the method of embodiment 2, wherein the normal butane stream is fed into a maleic anhydride unit. Embodiment 8 is the method of embodiment 2, wherein the hydrocarbon feed is crude oil. Embodiment 9 is the method of embodiment 2, wherein the reaction conditions in the isobutane dehydrogenation unit include a pressure from 0.5 to 15 psia and temperature from 550° C. to 700° C. Embodiment 10 is the method of embodiment 2, wherein the reaction conditions during hydrogenation include a pressure in the range of from 22 to 32 bara and temperature from 50 to 125° C. The hydrogen to feed ratio of 0.01 to 0.03 wt./wt. Embodiment 11 is a method for preparing methyl tert-butyl ether (MTBE), the method including the steps of separating from a hydrocarbon feed a gaseous stream having a boiling point of less than 200° C., a bottom stream containing fuel and pitch, and a crude C4 hydrocarbon stream containing C4 hydrocarbons from, wherein the crude C4 hydrocarbon stream containing normal butanes and isobutane; feeding the gaseous stream to a steam cracker and reacting the gaseous stream in the steam cracker with a steam cracking catalyst to form a reaction product containing C4 olefins and unreacted C4 paraffins; separating from the reaction product the C4 olefins and the unreacted C4 paraffins to form a crude steam-cracked C4 stream containing the C4 olefins and the unreacted C4 paraffins, and feeding the crude steam-cracked C4 stream to a hydrogenation unit; hydrogenating the crude steam-cracked C4 stream in the hydrogenation unit to hydrogenate the C4 olefins to form C4 paraffins and produce a hydrogenated C4 stream containing isobutane and normal butane; combining the hydrogenated C4 stream with the crude C4 hydrocarbon stream to form a combined C4 paraffin stream; feeding the combined C4 paraffin stream into a deisobutanizer unit to form an isobutane stream, a normal butane stream containing the normal C4 butane, and a bottom stream containing C5+ hydrocarbons; feeding the isobutane stream to an isobutane dehydrogenation unit under process conditions sufficient to dehydrogenate the isobutane to form a C4 hydrocarbon mixture containing isobutene and unreacted C4 hydrocarbons; feeding the C4 hydrocarbon mixture to an MTBE synthesis unit; and reacting the isobutene with methanol in the presence of a catalyst in the MTBE synthesis unit to form MTBE; separating the methyl tertiary butyl ether from the unreacted portion of the distillate stream, the unreacted portion containing isobutane and 1-butene; combining the normal butane stream and the bottom stream containing C5+ hydrocarbons to form a combined C4/C5+ stream and feeding the combined C4/C5+ stream to the steam cracker. Embodiment 12 is the method of embodiment 11, wherein the hydrocarbon feed is crude oil. Embodiment 13 is the method of embodiment 11, wherein the reaction conditions in the isobutane dehydrogenation unit include a pressure from 0.5 to 15 psia and temperature from 550° C. to 700° C. Embodiment 14 is the method of embodiment 11, wherein the reaction conditions during hydrogenation include wherein the reaction conditions during hydrogenation include a pressure in the range of from 22 to 32 bara and temperature from 50 to 125° C. The hydrogen to feed ratio of 0.01 to 0.03 wt./wt. Embodiment 15 is a method for preparing methyl tert-butyl ether (MTBE), the method including the steps of separating from a hydrocarbon feed a gaseous stream having a boiling point of less than 200° C., a bottom stream containing fuel and pitch, and a crude C4 hydrocarbon stream containing C4 hydrocarbons from, wherein the crude C4 hydrocarbon stream contains normal butanes and isobutane; feeding the gaseous stream to a steam cracker and reacting the gaseous stream in the steam cracker with a steam cracking catalyst to form a reaction product containing olefins, wherein the olefins contains C4 olefins; separating the C4 olefins to form a C4 olefin stream and feeding the C4 olefin stream into a hydrogenation unit; hydrogenating the C4 olefin stream with hydrogen under conditions such that the C4 olefins are hydrogenated to form C4 paraffins containing isobutane and normal butane; combining the C4 paraffins with the fresh C4 hydrocarbon stream to form a combined C4 stream; feeding the combined C4 hydrocarbon stream to a deisobutanizer unit to form an isobutane stream, a normal butane stream containing the normal C4 butanes, and a bottom stream containing C5+ hydrocarbons; feeding the isobutane stream to an isobutane dehydrogenation unit under process conditions sufficient to dehydrogenate the isobutane to form a C4 hydrocarbon mixture containing isobutene and unreacted C4 hydrocarbons; feeding the C4 hydrocarbon mixture to an MTBE synthesis unit; and reacting the isobutene with methanol in the presence of a catalyst in the MTBE synthesis unit to form MTBE; separating the methyl tertiary butyl ether from the unreacted portion of the distillate stream, the unreacted portion containing isobutane and 1-butene; and combining the normal butane stream and the bottom stream containing C5+ hydrocarbons to form a combined C4/C5+ stream and feeding the combined C4/C5+ stream to the steam cracker. Embodiment 16 is the method of embodiment 15, wherein the hydrocarbon feed is crude oil. Embodiment 17 is the method of embodiment 15, wherein the reaction conditions in the isobutane dehydrogenation unit include wherein the reaction conditions in the isobutane dehydrogenation unit include a pressure from 0.5 to 15 psia and temperature from 550° C. to 700° C. Embodiment 18 is the method of embodiment 15, wherein the reaction conditions during hydrogenation include a pressure in the range of from 22 to 32 bara and temperature from 50 to 125° C. The hydrogen to feed ratio of 0.01 to 0.03 wt./wt. Embodiment 19 is the method of any of the preceding embodiments, wherein the conditions in the maleic anhydride unit temperatures of 375° C. to 425° C.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A method for preparing methyl tert-butyl ether (MTBE), the method comprising the steps of:
separating from a hydrocarbon feed a gaseous stream having a boiling point of less than 200° C., a bottom stream comprising fuel and pitch, and a crude C4 hydrocarbon stream comprising C4 hydrocarbons from, wherein the crude C4 hydrocarbon stream comprises normal butanes and isobutane;
feeding the gaseous stream to a steam cracker and reacting the gaseous stream in the steam cracker with a steam cracking catalyst to form a reaction product comprising C4 olefins and unreacted C4 paraffins;
separating from the reaction product the C4 olefins and the unreacted C4 paraffins to form a crude steam-cracked C4 stream containing the C4 olefins and the unreacted C4 paraffins, and feeding the crude steam-cracked C4 stream to a hydrogenation unit;
hydrogenating the crude steam-cracked C4 stream in the hydrogenation unit to hydrogenate the C4 olefins to form C4 paraffins and produce a hydrogenated C4 stream comprising isobutane and normal butane;
combining the hydrogenated C4 stream with the crude C4 hydrocarbon stream to form a combined C4 paraffin stream;
feeding the combined C4 paraffin stream into a deisobutanizer unit to form an isobutane stream, a normal butane stream containing the normal C4 butane, and optionally a bottom stream comprising C5+ hydrocarbons;
feeding the isobutane stream to an isobutane dehydrogenation unit under process conditions sufficient to dehydrogenate the isobutane to form a C4 hydrocarbon mixture comprising isobutene and unreacted C4 hydrocarbons;
feeding the C4 hydrocarbon mixture to an MTBE synthesis unit; and
reacting the isobutene with methanol in the presence of a catalyst in the MTBE synthesis unit to form MTBE; and
separating the methyl tertiary butyl ether from the unreacted portion of the distillate stream, the unreacted portion comprising isobutane and 1-butene.

2. The method of claim 1, wherein the normal butane stream is fed into a maleic anhydride unit.

3. The method of claim 1, wherein the hydrocarbon feed is crude oil.

4. The method of claim 1, wherein the reaction conditions in the isobutane dehydrogenation unit include a pressure from 0.5 to 15 psia and temperature from 550° C. to 700° C.

5. The method of claim 1, wherein the reaction conditions during hydrogenation include a pressure in the range of from 22 to 32 bara and temperature from 50 to 125° C. The hydrogen to feed ratio of 0.01 to 0.03 wt./wt.

6. A method for preparing methyl tert-butyl ether (MTBE), the method comprising the steps of:
separating from a hydrocarbon feed a gaseous stream having a boiling point of less than 200° C., a bottom stream comprising fuel and pitch, and a crude C4 hydrocarbon stream comprising C4 hydrocarbons from, wherein the crude C4 hydrocarbon stream comprises normal butanes and isobutane;
feeding the gaseous stream to a steam cracker and reacting the gaseous stream in the steam cracker with a steam cracking catalyst to form a reaction product comprising olefins, wherein the olefins comprise C4 olefins;
separating the C4 olefins to form a C4 olefin stream and feeding the C4 olefin stream into a hydrogenation unit;
hydrogenating the C4 olefin stream with hydrogen under conditions such that the C4 olefins are hydrogenated to form C4 paraffins comprising isobutane and normal butane;
combining the C4 paraffins with the fresh C4 hydrocarbon stream to form a combined C4 stream;
feeding the combined C4 hydrocarbon stream to a deisobutanizer unit to form an isobutane stream, a normal butane stream containing the normal C4 butanes, and a bottom stream comprising C5+ hydrocarbons;
feeding the isobutane stream to an isobutane dehydrogenation unit under process conditions sufficient to dehydrogenate the isobutane to form a C4 hydrocarbon mixture comprising isobutene and unreacted C4 hydrocarbons;
feeding the C4 hydrocarbon mixture to an MTBE synthesis unit; and
reacting the isobutene with methanol in the presence of a catalyst in the MTBE synthesis unit to form MTBE; and
separating the methyl tertiary butyl ether from the unreacted portion of the distillate stream, the unreacted portion comprising isobutane and 1-butene.

7. The method of claim 2, wherein the normal butane stream is fed into a maleic anhydride unit, and/or
wherein the hydrocarbon feed is crude oil, and/or
wherein the reaction conditions in the isobutane dehydrogenation unit include a pressure from 0.5 to 15 psia and temperature from 550° C. to 700° C., and/or
wherein the reaction conditions during hydrogenation include a pressure in the range of from 22 to 32 bara and temperature from 50 to 125° C. and/or the hydrogen to feed ratio of 0.01 to 0.03 wt./wt.

8. A method for preparing methyl tert-butyl ether (MTBE), the method comprising the steps of:
separating from a hydrocarbon feed a gaseous stream having a boiling point of less than 200° C., a bottom stream comprising fuel and pitch, and a crude C4 hydrocarbon stream comprising C4 hydrocarbons from, wherein the crude C4 hydrocarbon stream comprises normal butanes and isobutane;
feeding the gaseous stream to a steam cracker and reacting the gaseous stream in the steam cracker with a steam cracking catalyst to form a reaction product comprising C4 olefins and unreacted C4 paraffins;
separating from the reaction product the C4 olefins and the unreacted C4 paraffins to form a crude steam-cracked C4 stream containing the C4 olefins and the unreacted C4 paraffins, and feeding the crude steam-cracked C4 stream to a hydrogenation unit;
hydrogenating the crude steam-cracked C4 stream in the hydrogenation unit to hydrogenate the C4 olefins to form C4 paraffins and produce a hydrogenated C4 stream comprising isobutane and normal butane;
combining the hydrogenated C4 stream with the crude C4 hydrocarbon stream to form a combined C4 paraffin stream;
feeding the combined C4 paraffin stream into a deisobutanizer unit to form an isobutane stream, a normal butane stream containing the normal C4 butane, and a bottom stream comprising C5+ hydrocarbons;
feeding the isobutane stream to an isobutane dehydrogenation unit under process conditions sufficient to dehydrogenate the isobutane to form a C4 hydrocarbon mixture comprising isobutene and unreacted C4 hydrocarbons;
feeding the C4 hydrocarbon mixture to an MTBE synthesis unit; and
reacting the isobutene with methanol in the presence of a catalyst in the MTBE synthesis unit to form MTBE;
separating the methyl tertiary butyl ether from the unreacted portion of the distillate stream, the unreacted portion comprising isobutane and 1-butene;
combining the normal butane stream and the bottom stream comprising C5+ hydrocarbons to form a combined C4/C5+ stream and feeding the combined C4/C5+ stream to the steam cracker.

9. The method of claim 8, wherein the hydrocarbon feed is crude oil and/or
wherein the reaction conditions in the isobutane dehydrogenation unit include a pressure from 0.5 to 15 psia and temperature from 550° C. to 700° C.

10. The method of claim 8, wherein the reaction conditions during hydrogenation include wherein the reaction conditions during hydrogenation include a pressure in the range of from 22 to 32 bara and temperature from 50 to 125° C. The hydrogen to feed ratio of 0.01 to 0.03 wt./wt.

11. A method for preparing methyl tert-butyl ether (MTBE), the method comprising the steps of:
separating from a hydrocarbon feed a gaseous stream having a boiling point of less than 200° C., a bottom stream comprising fuel and pitch, and a crude C4 hydrocarbon stream comprising C4 hydrocarbons from, wherein the crude C4 hydrocarbon stream comprises normal butanes and isobutane;
feeding the gaseous stream to a steam cracker and reacting the gaseous stream in the steam cracker with a steam cracking catalyst to form a reaction product comprising olefins, wherein the olefins comprise C4 olefins;
separating the C4 olefins to form a C4 olefin stream and feeding the C4 olefin stream into a hydrogenation unit;
hydrogenating the C4 olefin stream with hydrogen under conditions such that the C4 olefins are hydrogenated to form C4 paraffins comprising isobutane and normal butane;
combining the C4 paraffins with the fresh C4 hydrocarbon stream to form a combined C4 stream;
feeding the combined C4 hydrocarbon stream to a deisobutanizer unit to form an isobutane stream, a normal butane stream containing the normal C4 butanes, and a bottom stream comprising C5+ hydrocarbons;
feeding the isobutane stream to an isobutane dehydrogenation unit under process conditions sufficient to dehydrogenate the isobutane to form a C4 hydrocarbon mixture comprising isobutene and unreacted C4 hydrocarbons;
feeding the C4 hydrocarbon mixture to an MTBE synthesis unit; and
reacting the isobutene with methanol in the presence of a catalyst in the MTBE synthesis unit to form MTBE;
separating the methyl tertiary butyl ether from the unreacted portion of the distillate stream, the unreacted portion comprising isobutane and 1-butene; and
combining the normal butane stream and the bottom stream comprising C5+ hydrocarbons to form a combined C4/C5+ stream and feeding the combined C4/C5+ stream to the steam cracker.

12. The method of claim 11, wherein the hydrocarbon feed is crude oil and/or
wherein the reaction conditions in the isobutane dehydrogenation unit include wherein the reaction conditions in the isobutane dehydrogenation unit include a pressure from 0.5 to 15 psia and temperature from 550° C. to 700° C.

13. The method of claim 15, wherein the reaction conditions during hydrogenation include a pressure in the range of from 22 to 32 bara and temperature from 50 to 125° C. The hydrogen to feed ratio of 0.01 to 0.03 wt./wt.

14. The method of claim 1, wherein the conditions in the maleic anhydride unit temperatures of 375° C. to 425° C.
